(19) United States
(12) Patent Application Publication
(10) Pub. No.: US 2014/0314844 A1
(43) Pub. Date: Oct. 23, 2014

(54) ORAL COMPLEX FORMULATION COMPRISING OMEGA-3 FATTY ACID AND HMG-COA REDUCTASE INHIBITOR WITH IMPROVED STABILITY

(71) Applicant: HANMI PHARM. CO., LTD, Hwaseong-si, Gyeonggi-do (KR)

(72) Inventors: Jin Cheul Kim, Seoul (KR); Jae Ho Kim, Suwon-si (KR); Eun Jin Yoon, Suwon-si (KR); Yong Il Kim, Suwon-si (KR); Jae Hyun Park, Suwon-si (KR); Jong Soo Woo, Suwon-si (KR)

(73) Assignee: HANMI PHARM. CO., LTD, Hwaseong-si, Gyeonggi-do (KR)

(21) Appl. No.: 14/358,853

(22) PCT Filed: Nov. 16, 2012
(86) PCT No.: PCT/KR2012/009718

§ 371 Date: May 16, 2014

(30) Foreign Application Priority Data
Nov. 17, 2011 (KR) 10-2011-0120493
Nov. 14, 2012 (KR) 10-2012-0128926

(51) Int. Cl. A61K 9/48; A61K 31/505; A61K 31/40; A61K 31/232

(52) U.S. Cl. CPC A61K 9/4891; A61K 31/232; A61K 31/505; A61K 31/40
USPC 424/463; 514/275; 514/423; 427/2.14

(57) ABSTRACT

The present invention relates to an oral complex formulation comprising omega-3 fatty acid or its derivatives encapsulated in a hard or soft capsule containing sorbitol and sorbitan, as well as an HMG-CoA reductase inhibitor. The formulation of the present invention comprising omega-3 fatty acid encapsulated with sorbitol and sorbitan provides better prevention related materials from being formed, leading to improved long-term storage stability. The oral complex formulation of the present invention also can raise the serum HDL-cholesterol level, while reducing both LDL-cholesterol and TG levels. The oral complex formulation of the present invention is useful for treatment of hyperlipidemia.

### FIELD OF THE INVENTION

The present invention relates to an oral complex formulation comprising an omega-3 fatty acid charged in a hard or soft capsule, and an HMG-CoA reductase inhibitor.

### BACKGROUND OF THE INVENTION

Marine oils, also commonly referred to as fish oils, are the main sources of omega-3 fatty acids which modulate lipid metabolism, e.g., eicosapentaenoic acid (EPA) and docosahexaenoic acid (DHA). Omega-3 fatty acids can, without adverse side effects, reduce the levels of serum triglycerides (TG) and serum low-density lipoprotein (LDL) cholesterol, lower systolic and diastolic blood pressures, and the heart rate, and repress the activation of a phospholipids complex, a blood coagulation factor.

Those omega-3 fatty acids currently available as prescription medicine are omega-3 fatty acid ethyl esters (hereinafter, referred to as “omega-3 fatty acid esters”). In other words, they are ethyl-esterified compounds of omega-3 fatty acids, which are polyunsaturated fatty acids obtained from fish oils containing DHA and EPA, and sold under the trademark OMACOR®. Such omega-3 fatty acid esters are generally formulated into capsule forms such as gelatin capsules, as disclosed in U.S. Pat. Nos. 5,502,077, 5,656,667 and 5,698,594.

Meanwhile, 3-hydroxy-3-methylglutaryl-coenzyme A (HMG-CoA) reductase inhibitors contain 3-hydroxy lactones or corresponding ring-opened dihydroxy acids, and are often referred to as “statins.”

Currently, different types of synthetic and semisynthetic HMG-CoA reductase inhibitors including simvastatin (ZOCOR®; see U.S. Pat. No. 4,444,784), pravastatin sodium salt (PRAVACHOL®; see U.S. Pat. No. 4,346,227), fluvastatin sodium salt (LESCOL®; see U.S. Pat. No. 5,354,772), atorvastatin calcium salt (LIPITOR®; see U.S. Pat. No. 5,273,995), cerivastatin sodium salt (also known as rivastatin; see U.S. Pat. No. 5,177,080), rosuvastatin calcium salt (CRESTOR®; see KR. Patent No. 105431) and pitavastatin calcium salt (LIVARO®; see KR Patent No. 101149) are used to control high level of serum cholesterol.

Statins have been typically used for maintaining cholesterol levels within the normal range. Statins lower cholesterol by decreasing the production of cholesterol through inhibition of HMG-CoA reductase which regulates the rate of cholesterol synthesis in the body, or by enhancing the capacity of the liver to remove low-density lipoprotein (LDL)-cholesterol already present in blood. Thus, the main function of the statins is diminishing LDL cholesterol. Statins are known to reduce the risk of coronary heart disease (CHD) to one third, yet have limited effects on TG and serum HDL.

However, excessive amounts of both LDL and TG are found in hypercholesterolemia and mixed dyslipidemia. and monotherapy of omega-3 fatty acid esters or statin in treating these diseases suffered from their its low efficacy. In contrast, combination therapies of omega-3 fatty acid esters with statins for hyperlipidemia have the advantages of being capable of raising serum HDL cholesterol, while reducing LDL-cholesterol and TG. Hence, there has been much research on a combined formulation of omega-3 fatty acid esters and other drugs. However, there is a disadvantage that the drug stability between the two different drugs cannot be guaranteed when directly mixed.

For these reasons, the inventors of the present invention have conducted researches on an oral complex formulation comprising omega-3 fatty acids and an HMG-CoA reductase inhibitor. Glycerol is typically used as a plasticizer in preparation of a soft or hard capsule formulation. However, the inventors of the present invention discovered an unexpected phenomenon in which a specific, related material of formula (I) below was increased when a formulation was stored under accelerated conditions, the formulation comprising a layer comprising an HMG-CoA reductase inhibitor (e.g., rosuvastatin) coated on a capsule core containing omega-3 fatty acid ester and glycerol:

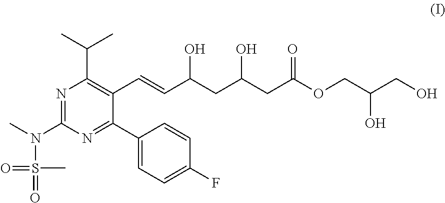 [SMILES: CC(C)c1nc(N(C)S(C)(=O)=O)nc(-c2ccc(F)cc2)c1/C=C/C(O)CC(O)CC(=O)OCC(O)CO]

Therefore, the present inventors have endeavored to develop a method for improving stability of the complex formulation in place of using glycerol or its derivatives. These efforts resulted in a formulation which comprises core encapsulating an omega-3 fatty acid with sorbitol and sorbitan in a hard or soft capsule, wherein the core is coated with a separated coating containing a water-resistant material, which is further coated with a drug layer comprising an HMG-CoA reductase inhibitor and a basic stabilizer. The formulation of the present invention has improved stability, showing good inhibitory effect on the production of the related materials.

### SUMMARY OF THE INVENTION

Accordingly, it is an object of the present invention to provide an oral complex formulation with improved long-term storage stability, comprising an omega-3 fatty acid and an HMG-COA reductase inhibitor.

It is another object of the present invention to provide a method for preparing the oral complex formulation.

In accordance with one aspect of the present invention, there is provided an oral complex formulation comprising:

(1) a capsule core comprising a hard or soft capsule containing sorbitol, sorbitan and as a pharmaceutically effective component, an omega-3 fatty acid;

(2) a first coating layer comprising a water-resistant coating material, which is formed on the surface of the capsule core; and

(3) a second coating layer comprising an HMG-CoA reductase inhibitor and a basic stabilizer, which is formed on the surface of the first coating layer

In accordance with another aspect of the present invention, there is provided a method for preparing the oral complex formulation comprising the steps of:

i) filling a hard or soft capsule comprising sorbitol and sorbitan with an omega-3 fatty acid to prepare a capsule core;

ii) forming a first coating layer comprising a water-resistant coating material on the surface of the capsule core; and

iii) forming a second coating layer comprising an HMG-CoA reductase inhibitor and a basic stabilizer on the surface of the first coating layer.

### BRIEF DESCRIPTION OF THE DRAWINGS

The above and other objects and features of the present invention will become apparent from the following descriptions of the invention, when taken in conjunction with the accompanying drawings, which respectively show:

FIG. 1: a graph comparing production rates of related materials depending on capsule compositions.

FIGS. 2 and 3: the rates of production (%) of related materials for the rosuvastatin-containing complex formulations prepared in Example 2 and Comparative Example 2, respectively.

FIGS. 4 and 5: the rates of production (%) of related materials for the atorvastatin-containing complex formulations prepared in Comparative Examples 3 and 4, and Examples of 3 and 4, respectively; and

FIGS. 6 and 7: a graph showing the increases in the lactone and unknown related materials produced, respectively, for Examples 5 to 8 and Comparative Examples 5 and 6.

### DETAILED DESCRIPTION OF THE INVENTION

The present invention provides an oral complex formulation comprising: (1) a capsule core comprising a hard or soft capsule containing sorbitol, sorbitan and as a pharmaceutically effective component, an omega-3 fatty acid; (2) a first coating layer comprising a water-resistant coating material, which is formed on the surface of the capsule core; and (3) a second coating layer comprising an HMG-CoA reductase inhibitor and a basic stabilizer, which is formed on the surface of the first coating layer.

Hereinafter, the components contained in the oral complex formulation of the present invention are described in detail.

### (a) Capsule Core

The complex formulation of the present invention comprises a capsule core comprising a hard or soft capsule containing sorbitol, sorbitan and as a pharmaceutically effective component, an omega-3 fatty acid. In an embodiment of the present invention, the complex formulation of the present invention comprises a hard or soft capsule comprising sorbitol and sorbitan, and as a pharmaceutically effective component, an omega-3 fatty acid contained in the capsule.

The amount of sorbitol may ranges 1 to 40% by weight, preferably 5 to 30% by weight, more preferably 7 to 25% by weight, based on the total weight of the hard or soft capsule. Also, the amount of sorbitan may ranges from 1 to 45% by weight, preferably 3 to 35% by weight, more preferably 6 to 30% by weight, based on the total weight of the hard or soft capsule. In one embodiment of the present invention, sorbitol may be contained in an amount ranging from 14 to 15% by weight, for instance about 14.5% by weight, and sorbitan may be contained in an amount ranging from 11 to 12% by weight, for instance about 11.6% by weight, based on the total weight of the hard or soft capsule.

When the hard or soft capsule comprises sorbitol and sorbitan in the above-described ranges, the storage stability of the complex formulation is improved due to significant reduction in the related materials produced during long-term storage under accelerated conditions.

In one embodiment of the present invention, sorbitol and sorbitan may be employed in the form of a solution containing them (hereinafter, “sorbitol sorbitan solution”). For instance, sorbitol sorbitan solution prepared by mixing sorbitol and sorbitan in a solvent such as distilled water may be employed in the preparation of the hard or soft capsule.

In accordance with USP-NF standard, sorbitol and sorbitan may be contained in an amount of at least 25% by weight, and at least 15% by weight, respectively, based on the total weight of the sorbitol sorbitan solution. For example, sorbitol may be contained in an amount ranging from 25 to 30% by weight, based on the total weight of the sorbitol sorbitan solution, whereas sorbitan may be contained in an amount ranging from 15 to 42% by weight based on the total weight of the sorbitol sorbitan solution.

In the present invention, the sorbitol sorbitan solution may be contained in an amount ranging from 20 to 70% by weight, e.g., 30 to 60% by weight, based on the total weight of the hard or soft capsule.

In one embodiment of the present invention, the hard or soft capsule comprising sorbitol and sorbitan may have a weight ratio of gelatin to the sorbitol sorbitan solution in the range of 1:0.4 to 1:1.2, e.g., about 1:0.7.

The hard or soft capsule of the present invention may comprise glycerol or a derivative thereof in an amount ranging from 0 to 20% by weight based on the total weight of the capsule.

Generally, gelatin is used as a capsule material so as to enhance elasticity for easier shaping. Also, a plasticizer may be employed in the capsule material or externally applied to the capsule so as to prevent possible damage caused from cast or change in shape when stored. Examples of the plasticizer may include glycerol or a derivative thereof, e.g., propylene glycol, polyethylene glycol (PEG), medium-chain triglyceride (MCT) oils, and the like.

The hard or soft capsule used in the present invention can be a conventional hard or soft capsule having gelatin as the main ingredient, which may comprise Pullulan or hydroxypropyl methylcellulose (HPMC); or it may not comprise or may comprise glycerol or the derivative thereof (e.g. propylene glycol, polyethylene glycol, medium-chain triglyceride oils, or a derivative thereof) in an amount of at most 20% by weight, e.g., 0.1 to 20% by weight, based on the total weight of the capsule.

In the present invention, “omega-3 fatty acids” may be natural or synthetic omega-3 fatty acid and encompasses all possible forms thereof. For instance, derivatized forms of free acid of omega-3 fatty acid, e.g., a pharmaceutically acceptable ester, a derivative, a precursor, a salt, or any mixture of the foregoing, as well as underivatized forms (i.e., a free acid) of omega-3 fatty acid may be used in the present invention. Specific but non-limiting examples of the omega-3 fatty acid are eicosapenta-5,8,11,14,17-enoic acid (eicosapentaenoic acid, EPA), docosahexa-4,7,10,13,16,19-enoic acid (docosahexaenoic acid, DHA) and long chain omega-3 polyunsaturated fatty acids (such as α-linolenic acid). Some examples of esters of omega-3 fatty acid that can be used in the present invention are those with glycerol (such as mono-, di- and triglycerides) and primary alcohols (such as methyl and ethyl esters of omega-3 fatty acid). Some examples of precursors of omega-3 fatty acid include precursors of corresponding omega-3 fatty acid oils (such as precursors of EPA, DHA and α-linolenic acid). Some examples of derivatives of omega-3 fatty acid include polysaccharide derivatives and polyoxyethylene derivatives of omega-3 fatty acids. In an embodiment, the omega-3 fatty acid may be selected from the group consisting of EPA, DHA, triglycerides of EPA or DHA, ethyl esters of EPA or DHA, and a mixture thereof.

The omega-3 fatty acid, the pharmaceutically acceptable ester, derivative, precursor, or salt thereof, a mixture thereof may be used in their pure forms or included in fish oil, preferably a highly purified fish oil concentrate, perilla oil or marine microalgae oil containing them.

In the present invention, the omega-3 fatty acid may be contained in an amount ranging from 70 to 95% by weight, based on the total weight of the capsule core.

Unlike a conventional hard or soft capsule containing glycerol or a derivative thereof as a plasticizer, the hard or soft capsule used in the present invention may be prepared in accordance with a conventional method for preparing a capsule by employing no glycerol or a derivative thereof or employing glycerol or a derivative thereof with only a small amount e.g., in an amount at most 20% by weight based on the total weight of the capsule, while comprising sorbitol and sorbitan. The capsule thus obtained may be filled with an omega-3 fatty acid to obtain a capsule core.

### (b) First Coating Layer

In the oral complex formulation of the present invention, the first coating layer comprises a water-resistant coating material which encapsulates the capsule core in order to prevent various ingredients including water content of gelatin within the capsule core from affecting the dissolution rate of an HMG-CoA reductase inhibitor-containing second coating layer and production of related materials.

Specifically, when an HMG-CoA reductase inhibitor is coated directly on the capsule core containing omega-3 fatty acids, then water content of the gelatin capsule can affect the content of the HMG-CoA reductase inhibitor, reduce its dissolution rate and increase its related materials. In the present invention, however, the first coating layer comprising a water-resistant coating material is formed between the omega-3 fatty acid-containing capsule core and the HMG-CoA reductase inhibitor-containing second coating layer, minimizing the effect of water content as well as other potential risks.

Examples of the water-resistant coating material may be selected from the group consisting of hydroxyethyl cellulose, hydroxypropyl cellulose, hydroxypropylmethyl cellulose, polyethylene glycol, polyvinylpyrrolidone, polyvinylpyrrolidone-vinyl acetate copolymer, ethyl cellulose and a mixture thereof, e.g., ethyl cellulose.

According to one embodiment of the present invention, the use of ethyl cellulose as the water-resistant coating materials resulted in significantly reduced amount of related material than the use of other materials (See Test Example 4).

The water-resistant coating material may be used in the amount ranging from 15 to 75% by weight, e.g., 16 to 72% by weight, based on the total amount of the first coating layer.

The first coating layer may be formed by dissolving or dispersing the water-resistant coating material in water, ethanol, or a mixture thereof (for example a mixed solvent of water and ethanol in a weight ratio of 1:1 to 1:3, e.g., about 3:7) to obtain a coating solution and then applying the solution onto the surface of the capsule core.

The first coating layer may be coated on the surface of the capsule core in an amount ranging from 1 to 20 parts by weight, e.g., 4 to 10 parts by weight, based on 100 parts by weight of the capsule core. If the amount of the first coating layer is less than 1 part by weight, the first coating layer becomes too thin, and thus affects the separation of two drugs and water transfer, which deteriorates the inhibition of the production of related materials. On the other hand, if the amount exceeds 20 parts by weight, the first coating layer becomes too thick, significantly reducing the effects of combination therapy with omega-3 fatty acid and HMG-CoA reductase inhibitor.

### (c) Second Coating Layer (HMG-CoA Reductase Inhibitor Coating Layer)

In the oral complex formulation of the present invention, the second coating layer comprises an HMG-CoA reductase inhibitor and a basic stabilizer, which is coated on the surface of the first coating layer.

The HMG-CoA reductase inhibitor blocks the reduction of HMG-CoA mevalonate, thereby reducing the levels of lipids and cholesterol in the blood, and thus it can be used for prevention and treatment of hyperlipidemia, hypercholesterolemia or atherosclerosis.

In the present invention, the HMG-CoA reductase inhibitor may be selected from the group consisting of simvastatin, pravastatin, fluvastatin, atorvastatin, cerivastatin, rosuvastatin, pitavastatin and a pharmaceutically acceptable salt thereof, e.g., rosuvastatin or atorvastatin. The HMG-CoA reductase inhibitors may be used in an amount of 0.05 to 20 parts by weight, preferably 0.1 to 8 parts by weight, based on 100 parts by weight of the capsule core.

One drawback of the HMG-CoA reductase inhibitor is that it easily undergoes a hydrolysis due to its chemical structure which yields the production of related materials having lactone structure as a hydrolysis product, thereby decreasing its efficacy. In the present invention, the second coating layer comprises a basic stabilizer which prevents hydrolysis of an HMG-CoA reductase inhibitor, thereby to inhibit the production of related materials. The oral complex formulation of the present invention comprises both an HMG-CoA reductase inhibitor and omega-3 fatty acids, and thus, it is important to select an ingredient which can inhibit the hydrolysis of the HMG-CoA reductase inhibitor to prevent the production of related materials.

The basic stabilizer used in the present invention may be selected from the group consisting of magnesium carbonate (MgCO₃), sodium hydrogen carbonate (NaHCO₃), magnesium hydroxide (Mg(OH)₂) and a mixture thereof, e.g., magnesium carbonate or sodium hydrogen carbonate. Sparingly soluble materials with unacceptable solubility for pharmaceutical compositions such as calcium carbonate (CaCO₃) are not preferred, despite being alkaline.

In the present invention, the basic stabilizer may be employed in an amount ranging from 0.01 to 40% by weight based on the total weight of the second coating layer. For example, magnesium carbonate may be employed in an amount ranging from 5 to 40% by weight, sodium hydrogen carbonate in an amount ranging from 0.01 to 2% by weight, and magnesium hydroxide in an amount ranging from 0.01 to 2% by weight, based on the total weight of the second coating layer.

In accordance with one embodiment of the present invention, certain materials that are similar to sodium hydrogen carbonate (e.g., calcium carbonate), were unable to effectively prevent the production of unknown related materials (see Test Example 3).

In the present invention, the second coating layer comprises an HMG-CoA reductase inhibitor and a basic stabilizer in a weight ratio of 0.1 to 200:1. For example, if the basic stabilizer is magnesium carbonate, the weight ratio of HMG-CoA reductase inhibitor:magnesium carbonate may be 0.1:1 to 3.0:1; in case of sodium hydrogen carbonate, the weight ratio of HMG-CoA reductase inhibitor:sodium hydrogen carbonate may be 10:1 to 100:1; and in case of magnesium hydroxide, the weight ratio of HMG-CoA reductase inhibitor:magnesium hydroxide may be 10:1 to 100:1.

Meanwhile, the second coating layer of the present invention may comprise a coating material selected from the group consisting of hydroxypropyl methylcellulose, polyvinylpyrrolidone, polyethylene glycol, polyvinyl alcohol-polyethylene glycol graft copolymer and a mixture thereof. In the present invention, the formulation can release the HMG-CoA reductase inhibitor rapidly by comprising the second coating layer.

In accordance with one embodiment of the present invention, the second coating layer may be formed by dissolving or dispersing an HMG-CoA reductase inhibitor as the second pharmaceutically active ingredient and a mixture of polyvinylpyrrolidone and polyvinyl alcohol-polyethylene glycol graft copolymer in water, ethanol, or a mixture thereof (for example, a mixed solvent of water and ethanol in a weight ratio of about 3:7) to obtain a coating solution, and then applying the solution onto the surface of the first coating layer.

In the present invention, the coating material may be employed in an amount ranging from 25 to 85% by weight, preferably 25 to 80% by weight, based on the total weight of the second coating layer.

The second coating layer may be coated on the surface of the first coating layer in an amount ranging from 3 to 30 parts by weight, preferably 5 to 20 parts by weight, e.g., 3 to 10 parts by weight, based on 100 parts by weight of the capsule core.

The oral complex formulation in accordance with the present invention may further comprise other pharmaceutically acceptable additives such as disintegrants, diluents, stabilizers, binders and lubricants, if necessary.

The present invention also provides a method for preparing the oral complex formulation of the present invention, which comprises the steps of: (i) filling a hard or soft capsule comprising sorbitol and sorbitan with omega-3 fatty acid to prepare a capsule core; (ii) forming a first coating layer comprising a water-resistant coating material on the surface of the core; and (iii) forming a second coating layer comprising an HMG-CoA reductase inhibitor and a basic stabilizer on the surface of the first coating layer.

In the method of the present invention, the second coating layer may further comprise hydroxypropyl methyl cellulose, polyvinylpyrrolidone, polyethylene glycol, polyvinyl alcohol-polyethylene glycol graft copolymer or a mixture thereof in the step (iii) above.

According to one embodiment of the present invention, the method for preparing the oral complex formulation of the present invention may comprise the following steps of: (1) preparing a capsule comprising sorbitol and sorbitan as a plasticizer in a conventional manner for manufacturing capsules, followed by filling the capsule with omega-3 fatty acid to obtain a capsule core; (2) forming a first coating layer encapsulating the capsule core by dissolving a water-resistant coating material in a suitable solvent (e.g. in a mixture of ethanol and water), applying the coating solution thus obtained onto the capsule core, and then drying the solution; and (3) forming a second coating layer on the first coating layer by dissolving a coating material such as hydroxypropyl methyl cellulose, polyvinylpyrrolidone, polyethylene glycol, polyvinyl alcohol-polyethylene glycol graft copolymer or a mixture thereof and an HMG-CoA reductase inhibitor, together with a basic stabilizer and any pharmaceutically acceptable additives in a suitable solvent (e.g., in a mixture of ethanol and water), applying the coating solution thus obtained onto the surface of the first coating layer, and then drying the solution. The complex formulation thus prepared may be formulated into a coated tablet form, and can be administered orally.

In step (1), the capsule may comprise glycerol or its derivative, which may be contained in an amount of at most 20% by weight, based on the total weight of the capsule.

The oral complex formulation prepared in accordance with the inventive method showed good long-term storage stability by meaningfully reducing the production of related materials during long-term storage under accelerated condition (40° C. and 75% RH) for 6 months.

The formulation of the present invention meets the ICH Guidelines requirement for unknown related material, i.e., less than 0.5% by weight. If the amount of unknown related material exceeds 0.5% by weight, the formulation is subjected to a number of toxicity tests requiring high costs such as repeated toxicity test, genotoxicity test and so on. However, the present invention is cost-effective because it yields formulations which produce smaller amount of unknown related materials without having to undergo additional toxicity tests.

The oral complex formulation according to the present invention comprises two (2) pharmaceutically active ingredients, i.e. omega-3 fatty acid and HMG-CoA reductase inhibitor, which can raise serum HDL cholesterol level while reducing both LDL-cholesterol and TG levels. Thus, it can be used for effectively preventing or treating hyperlipidemia, hypertriglyceridemia, hypercholesteremia, coronary arterial heart diseases (CHD), dyslipidemia, increased level of total serum cholesterol and increased level of serum LDL cholesterol (LDL-C) as well as decreased level of serum HDL cholesterol (HDL-C).

### EXAMPLES

The following Examples are provided to illustrate preferred embodiments of the present invention, and are not intended to limit the scope of the present invention.

### Reference Example

### Composition of Soft or Hard Capsule and Suitability Test Among Main Ingredients

In order to evaluate interaction between the ingredients of a soft or hard capsule and statins, the following test was performed.

Specifically, gelatin (grade: 165 bloom, Geltech, Korea) which is a main ingredient of capsule; glycerol, propylene glycol and polyethylene glycol (PEG400), which are often used as a plasticizer; and sorbitol sorbitan solution (ROQUETTE, France, NF grade, containing solid content of 27.5 wt % of sorbitan and 34.4 wt % of sorbitol) were examined to evaluate the interaction with rosuvastatin calcium. In accordance with the ingredients described in Table 1, all ingredients were mixed, heated at 100° C. for 2 hours, and then evaluated by HPLC analysis.

HPLC analysis was performed using a stainless steel column of approximately 250 mm length, packed with 5 μm C₁₈ or a similar column (Inertsil-ODS2, GL sciences) with a mobile phase of water:acetonitrile:1%(v/v) trifluoroacetic acid solution (62:37:1 (v/v)) at flow rate of 0.75 mL/min. The peak of related material as compared to the major peak of rosuvastatin, was analyzed quantitatively.

TABLE 1

| | Ref. | Ref. | Ref. | Ref. | Ref. | Ref. |
|---|---|---|---|---|---|---|
| | Ex. 1 | Ex. 2 | Ex. 3 | Ex. 4 | Ex. 5 | Ex. 6 |
| Rosuvastatin | 50 mg | 50 mg | 50 mg | 50 mg | 50 mg | 50 mg |
| Gelatin | — | 100 mg | — | — | — | — |
| Glycerol | — | — | 100 mg | — | — | — |
| Sorbitol Sorbitan | — | — | — | 100 mg | — | — |
| Solution | | | | | | |
| Propylene glycol | — | — | — | — | 100 mg | — |
| Polyethylene glycol | — | — | — | — | — | 100 mg |
| (PEG400) | | | | | | |

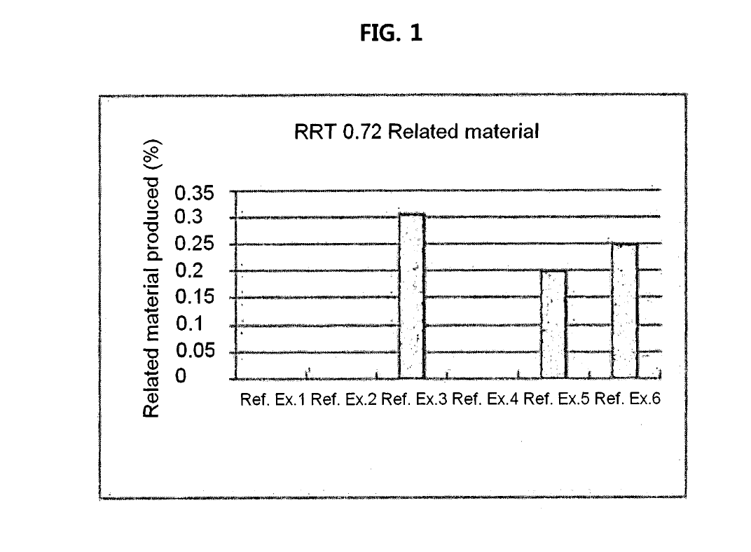

Summarizing the analysis results for Reference Examples 1 to 6, FIG. 1 compares the production rates (%) of one related material at issue (hereinafter, RRT 0.72) that rapidly increased among the various other related materials. As shown in FIG. 1, the related material of issue was produced from reactions with glycerol or glycerol derivatives in the rest of Reference Examples, but excluding Reference Examples 1 (containing rosuvastatin only), 2 (containing rosuvastatin without plasticizer), and 4 (containing rosuvastatin with sorbitol sorbitan solution). Thus, it suggests that the storage stability of rosuvastatin can be affected by different types of plasticizer.

In addition, the related material (RRT 0.72) was separated for further investigation and it was found that the related material was consistent with being a by-product of a reaction between rosuvastatin and glycerol. Based on the results above, the inventors employed the sorbitol sorbitan solution as an alternative to the conventional plasticizer, i.e., glycerol, to continue the following experiments.

### Examples 1-1 to 1-3 and Comparative Example 1

### Preparation of Capsule and Effects Depending on Capsule Composition

Due to the limitation on the amount allowed for the soft capsule material which will be used to encapsulate omega-3 fatty acid ester oils, soft capsules with different compositions were prepared and stability test was conducted in order to determine the proper mixture ratio between gelatin and the sorbitol sorbitan solution.

1) Preparation of Capsule

Capsules of Examples 1-1 to 1-3 and Comparative Example 1 were obtained by preparing a soft or hard gelatin capsule in accordance with the ingredients described in Table 2 using a conventional method, followed by filling the gelatin capsule thus obtained with 1,000 mg of omega-3 fatty acid ester oils (KD pharma, Germany, EP grade). The sorbitol sorbitan solution was employed as a plasticizer in the capsules of Examples 1-1 to 1-3, whereas glycerol was employed in the capsule of Comparative Example 1. Also, small amounts of glycine was added to the capsules of Examples 1-1 to 1-3 so as to prevent delayed disintegration.

TABLE 2

| | Amount (mg/capsule) | | | |
|---|---|---|---|---|
| | Comp. Ex. 1 | Ex. 1-1 | Ex. 1-2 | Ex. 1-3 |
| Gelatin | 293.0 | 246.8 | 296.8 | 196.8 |
| Glycerol | 135.0 | — | — | — |
| Sorbitol Sorbitan | — | 180.0 | 130.0 | 230.0 |
| Solution | | | | |
| Glycine | — | 1.2 | 1.2 | 1.2 |

2) Comparison of Stabilities in External Appearance after Drying and Accelerated Conditions Depending on Capsule Compositions

Stabilities in external appearance of soft capsules with different ratios of plasticizer were compared in the soft capsules prepared in Examples 1-1 to 1-3. The weight ratios of gelatin to plasticizer in Examples 1-1, 1-2 and 1-3 are approximately 1:0.7, 1:0.4 and 1:1.2, respectively.

It was observed that, during the preparation process of capsules according to Examples 1-1 to 1-3, if the ratio of gelatin to plasticizer was less than 1:0.4, cracks were found on the capsule. If the ratio exceeded 1:1.2, capsules were formed in irregular shape due to lack of gelatin, indicating the difficulty in the process of preparing soft capsules. Also, the capsules were stored under drying condition (25° C. and 15% RH) and accelerated condition (40° C. and 75% RH) for one week. As a result, it was observed that external appearance of the capsule of Example 1-1 showed less distortion, stretching, change in size and so on, as compared to the capsules of Examples 1-2 and 1-3.

Based on the results above, the soft capsule of Example 1-1 comprising the sorbitol sorbitan solution was employed to continue the following experiments.

3) Comparison of Disintegration Time According to Capsule Compositions

In order to analyze the disintegration time according to capsule compositions of a soft or hard capsule, the dissolution test was performed based on the dissolution test method described in Korean Pharmacopoeia using the capsules prepared in Example 1-1 and Comparative Example 1. As a result, the disintegration time of the capsules of Example 1-1 and Comparative Example 1 came out to be 9 min and 8 min 30 sec, respectively, which are both within the acceptable range of Korean Pharmacopoeia, regardless of the plasticizer type.

### Examples 2

### Preparation of Oral Complex Formulation Comprising Rosuvastatin

The first and the second coatings were applied in accordance with the ingredients described in Table 3 using the capsule of Example 1-1 as a capsule core. For the first coating layer, hydroxypropyl methylcellulose (HPMC), polyethylene glycol (PEG), polyvinylpyrrolidone (PVP) and ethylcellulose (Aqualon N7 grade, ASHLAND) were dissolved in a mixed solvent of ethanol and water in a weight ratio of 3:7 and a water-resistant coating was performed with the mixture thus obtained using a coating apparatus (Sejong, SFC-30). Subsequently, for the second coating layer, rosuvastatin calcium, a basic stabilizer (MgCO₃), polyvinylpyrrolidone and polyvinyl alcohol-polyethylene glycol graft copolymer (Kollicoat IR, BASF) were dissolved in a mixed solvent of ethanol and water in a weight ratio of 3:7 and a second coating was performed with the mixture thus obtained using a coating apparatus (Sejong, SFC-30), followed by drying to obtain the oral complex formulation.

TABLE 3

| The first coating layer | | The second coating layer | |
|---|---|---|---|
| HPMC 2910 (P645) | 74.0 mg | Rosuvastatin calcium | 10.4 mg |
| PEG 6000 | 10.0 mg | Kollicoat IR | 52.2 mg |
| PVP K-30 | 10.0 mg | PVP K-30 | 6.2 mg |
| Ethyl cellulose | 26.5 mg | MgCO₃ | 5.0 mg |
| (Ethanol) | 1036.0 mg | (Ethanol) | 460.0 mg |
| (Distilled water) | 444.0 mg | (Distilled water) | 460.0 mg |
| Total weight of coating | 120.5 mg | Total weight of coating | 73.8 mg |
| material | | material | |

### Comparative Examples 2

### Preparation of Oral Complex Formulation Comprising Rosuvastatin

The procedure of Example 2 was repeated except for using the capsule of Comparative Example 1 instead of the capsule of Example 1-1 to obtain the oral complex formulation of Comparative Example 2.

### Test Example 1

### Stability Test of Rosuvastatin

A stability test was performed to compare the production rate of related materials according to capsule compositions of the oral complex formulation comprising rosuvastatin.

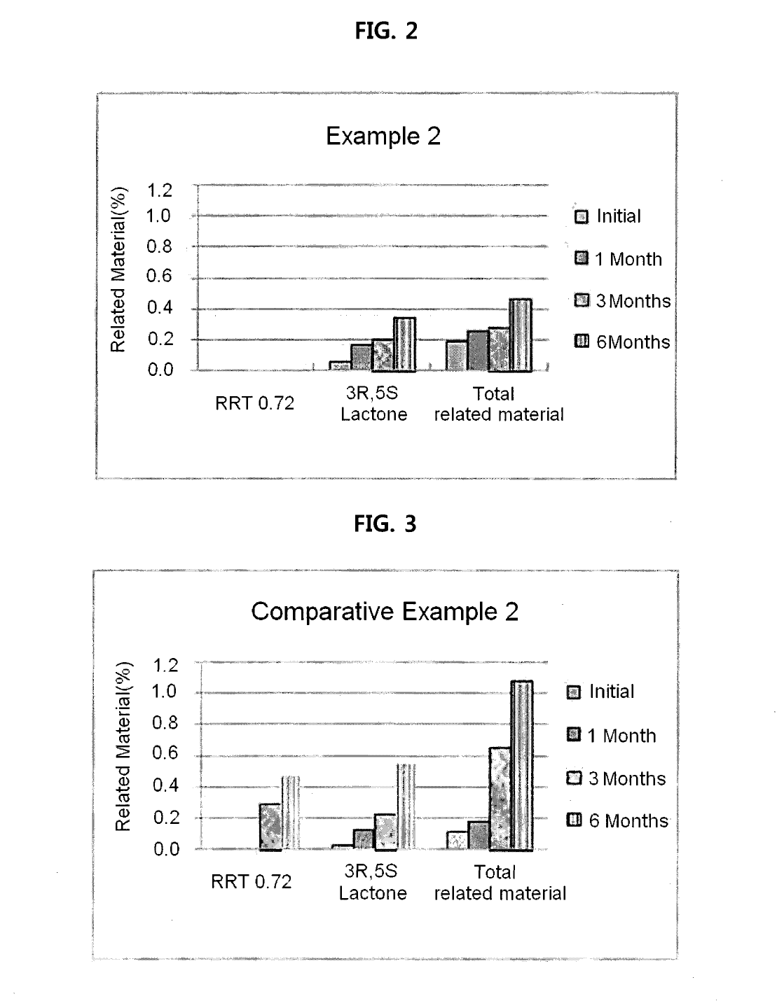

The oral complex formulations prepared in Example 2 and Comparative Example 2 were placed in sealed high-density polyethylene (HDPE) bottles and stored under accelerated storage condition (40° C. and 75% RH). Samples were taken at the initiation of the test and after 1, 3 and 6 months therefrom and then evaluated by HPLC analysis. HPLC analysis was performed using a stainless steel column of approximately 250 mm length, packed with 5 μm C₁₈ or a similar column (Inertsil-ODS2, GL sciences) with a mobile phase of water:acetonitrile:1% (v/v) trifluoroacetic acid solution (62:37:1 (v/v)) at the flow rate of 0.75 mL/min. The peaks of related materials as compared to the major peak of rosuvastatin were analyzed quantitatively. The results are shown in FIGS. 2 and 3.

As shown in FIGS. 2 and 3, the inventive complex formulation comprising sorbitol and sorbitan (Example 2) showed reduced amounts in RRT 0.72 related material, 3R,5S lactone-related material and total related materials as compared to the complex formulation of Comparative Example 2 containing glycerol, which suggests the inventive complex formulation has improved stability up to 6 months under accelerated storage conditions.

Particularly, the complex formulation of Example 2 demonstrated a noticeable reduction in production of RRT 0.72 related material, leading to a conclusion that it is appropriate to use an alternative plasticizer instead of glycerol in developing a rosuvastatin complex formulation.

### Examples 3 and 4

### Preparation of Oral Complex Formulation Comprising Atorvastatin

The first and the second coatings were applied in accordance with the ingredients described in Table 4 using the capsule of Example 1-1 as a capsule core. For the first coating layer, hydroxypropyl methylcellulose (HPMC), polyethylene glycol, polyvinylpyrrolidone and ethylcellulose were dissolved in a mixed solvent of ethanol and water in a weight ratio of 3:7, and a water-resistant coating was performed with the mixture thus obtained using a coating apparatus (Sejong, SFC-30). Subsequently, for the second coating layer, atorvastatin calcium, a basic stabilizer, polyvinylpyrrolidone and polyvinyl alcohol-polyethylene glycol graft copolymer were dissolved in a mixed solvent of ethanol and water in a weight ratio of 3:7 and a second coating was performed with the mixture thus obtained using a coating apparatus (Sejong, SFC-30), followed by drying to obtain the oral complex formulations of Examples 3 and 4. The formulations of Examples 3 and 4 comprise atorvastatin forms I and VIII, respectively.

TABLE 4

| The first coating layer | | The second coating layer | |
|---|---|---|---|
| HPMC 2910 (P645) | 74.0 mg | Atorvastatin calcium | 10.85 mg |
| PEG 6000 | 10.0 mg | Kollicoat IR | 52.2 mg |
| PVP K-30 | 10.0 mg | PVP K-30 | 6.0 mg |
| Ethyl cellulose | 26.5 mg | MgCO₃ | 40.0 mg |
| (Ethanol) | 1036.0 mg | (Ethanol) | 460.0 mg |
| (Distilled water) | 444.0 mg | (Distilled water) | 460.0 mg |
| Total weight of coating | 120.5 mg | Total weight of coating | 109.05 mg |
| material | | material | |

### Comparative Examples 3 and 4

### Preparation of Oral Complex Formulation Comprising Atorvastatin

The procedure of Example 3 was repeated except for using the capsule of Comparative Example 1 instead of the capsule of Example 1-1 to obtain the oral complex formulations of Comparative Examples 3 and 4. The formulations of Comparative Examples 3 and 4 comprise atorvastatin forms I and VIII, respectively.

### Test Example 2

### Stability Test of Atorvastatin

A stability test was performed to compare the production rate of related materials according to capsule compositions of the oral complex formulation comprising atorvastatin.

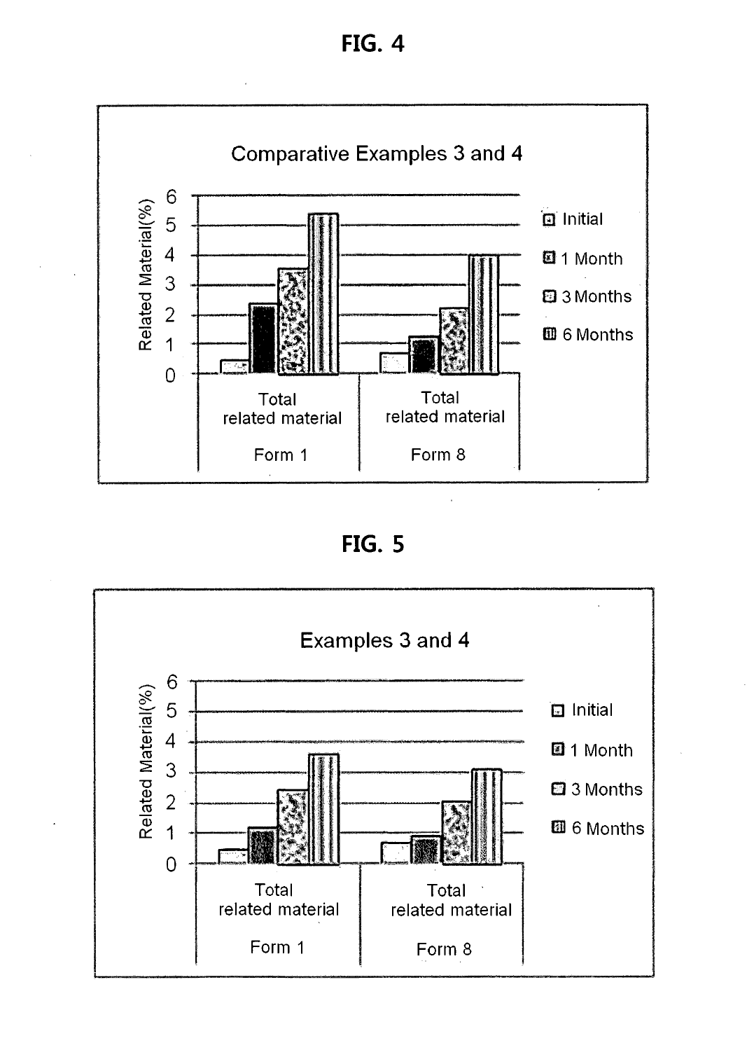

The oral complex formulations prepared in Example 3 and 4, and Comparative Examples 3 and 4 were placed in sealed high-density polyethylene (HDPE) bottles and stored under accelerated storage condition (40° C. and 75% RH). Samples were taken after the initiation of the test and after 1, 3 and 6 months therefrom, and related materials of atorvastatin were analyzed with reference to the atorvastatin calcium monograph of USP32. The results are shown in FIGS. 4 and 5.

As shown in FIGS. 4 and 5, the inventive complex formulations comprising sorbitol and sorbitan (Examples 3 and 4) showed reduced amount of the total related material as compared to Comparative Examples 3 and 4, suggesting that the inventive complex formulations has improved stability up to 6 months under accelerated storage conditions. It is expected that the increase of related material was due to the reaction between atorvastatin and glycerol in a similar manner as in Test Example 1.

Therefore, based on the results of rosuvastatin and atorvastatin of Test Examples 1 and 2, a similar outcome can be expected for studies of complex formulations comprising statins and any other drugs filled in a soft or hard capsule.

### Examples 5 to 8 and Comparative Examples 5 and 6

### Preparation of Oral Complex Formulation

A soft capsule was prepared in accordance with the ingredients described in Table 5 using gelatin, the sorbitol sorbitan solution used in the Reference Example and glycine in a conventional manner for manufacturing a soft capsule, and then omega-3 fatty acid ethyl ester was filled in the soft capsule thus obtained.

Hydroxypropyl methylcellulose (HPMC), polyethylene glycol (PEG), polyvinylpyrrolidone (PVP) and ethylcellulose were mixed with ethanol (450 mg) and water (1,100 mg), and the first coating was performed with the mixture thus obtained using a coating apparatus (Sejong, SFC-30), followed by drying to obtain the capsule coated with the first coating layer.

In order to form the second coating layer which encases the surface of the first coating layer, rosuvastatin (10 mg) was dissolved in Kollicoat IR and Povidone, followed by adding sodium hydrogen carbonate in an amount described in Table 5 below to prepare a drug coating solution. The capsule was coated using the drug coating solution in the same manner as described above to obtain the oral complex formulation.

The complex formulations of Examples 7 and 8 were prepared in the weight ratios of rosuvastatin:sodium hydrogen carbonate=10:1 and 100:1, respectively, per 1.25 mg of rosuvastatin, which is substantially the same ratio of rosuvastatin:sodium hydrogen carbonate as in the complex formulations of Examples 5 and 6.

The complex formulations of Comparative Examples 5 and 6 were prepared by repeating the above procedure except for using calcium carbonate instead of sodium hydrogen carbonate.

TABLE 5

| | | Amount (mg/capsule) | | | | | |
|---|---|---|---|---|---|---|---|
| | | | | | | Comp. | Comp. |
| | Ingredient | Ex. 5 | Ex. 6 | Ex. 7 | Ex. 8 | Ex. 5 | Ex. 6 |
| Soft gelatin | Omega-3 | 1000.0 | 1000.0 | 1000.0 | 1000.0 | 1000.0 | 1000.0 |
| capsule | fatty acid | | | | | | |
| | ester | | | | | | |
| | Gelatin | 246.8 | 246.8 | 246.8 | 246.8 | 246.8 | 246.8 |
| | Sorbitol | 180.0 | 180.0 | 180.0 | 180.0 | 180.0 | 180.0 |
| | sorbitan soln. | | | | | | |
| | Glycine | 1.2 | 1.2 | 1.2 | 1.2 | 1.2 | 1.2 |
| The first | HPMC 2910 | 74.0 | 74.0 | 74.0 | 74.0 | 74.0 | 74.0 |
| coating | PEG 6000 | 10.0 | 10.0 | 10.0 | 10.0 | 10.0 | 10.0 |
| layer | Povidone | 10.0 | 10.0 | 10.0 | 10.0 | 10.0 | 10.0 |
| | Ethylcellulose | 28.0 | 28.0 | 28.0 | 28.0 | 28.0 | 28.0 |
| The second | Rosuvastatin | 10.4 | 10.4 | 1.30 | 1.30 | 10.4 | 1.30 |
| coating | calcium | (10.0) | (10.0) | (1.25) | (1.25) | (10.0) | (1.25) |
| layer | (rosuvastatin) | | | | | | |
| | NaHCO₃ | 1.0 | 0.1 | 0.125 | 0.0125 | — | — |
| | CaCO₃ | — | — | — | — | 1.0 | 0.125 |
| | Mg(OH)₂ | — | — | — | — | — | — |
| | Kollicoat IR | 60.0 | 60.0 | 60.0 | 60.0 | 60.0 | 60.0 |
| | Povidone | 8.8 | 8.8 | 8.8 | 8.8 | 8.8 | 8.8 |

### Test Example 3

### Analysis of Related Materials Produced

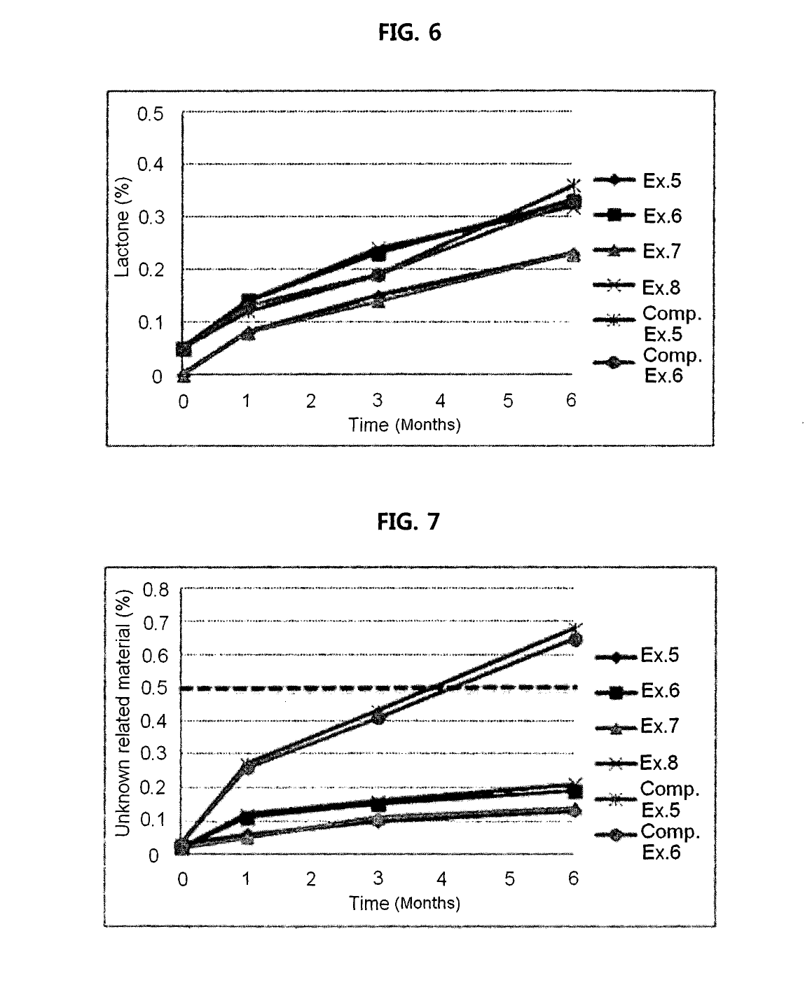

The oral complex formulations prepared in Examples 5 to 8 and Comparative Examples 5 and 6 were placed in sealed high-density polyethylene (HDPE) bottles and stored under accelerated storage condition (40° C. and 75% RH). Samples were taken after the initiation of the test and after 1, 3 and 6 months therefrom, and the production rate of related materials was analyzed in the same manner described in Test Example 1, and the results are shown in Tables 6 and 7 as well as FIGS. 6 and 7.

TABLE 6

| | Lactone related materials | | | |
|---|---|---|---|---|
| | Initial | 1 month | 3 months | 6 months |
| Ex. 5 | — | 0.08 | 0.15 | 0.23 |
| Ex. 6 | 0.05 | 0.12 | 0.23 | 0.37 |
| Ex. 7 | — | 0.08 | 0.14 | 0.23 |
| Ex. 8 | 0.05 | 0.12 | 0.24 | 0.38 |
| Comp. Ex. 5 | 0.05 | 0.12 | 0.19 | 0.36 |
| Comp. Ex. 6 | 0.05 | 0.13 | 0.17 | 0.33 |

TABLE 7

| | Unknown related materials | | | |
|---|---|---|---|---|
| | Initial | 1 month | 3 months | 6 months |
| Ex. 5 | 0.02 | 0.11 | 0.15 | 0.19 |
| Ex. 6 | 0.03 | 0.06 | 0.10 | 0.13 |
| Ex. 7 | 0.02 | 0.12 | 0.16 | 0.21 |
| Ex. 8 | 0.02 | 0.05 | 0.11 | 0.14 |
| Comp. Ex. 5 | 0.03 | 0.27 | 0.43 | 0.68 |
| Comp. Ex. 6 | 0.03 | 0.26 | 0.41 | 0.65 |

As shown in Tables. 6 and 7, complex formulations of Examples comprising sodium hydrogen carbonate as a basic stabilizer and Comparative Examples comprising calcium carbonate resulted in similar increase of lactone related material. However, the complex formulation of the present invention showed improved inhibitory effect on the production of unknown related materials.

FIGS. 6 and 7 represent the schematizations of Tables 6 and 7, wherein the dotted line in FIG. 7 indicates the ICH Guidelines requirement for unknown related material, i.e., less than 0.5% by weight. As shown in FIGS. 6 and 7, complex formulations of Comparative Examples 5 and 6 comprising calcium carbonate exceeded the limitations set forth by the ICH Guidelines after being stored for 6 months.

### Test Example 4

### Analysis of Related Material Produced Depending on the Presence of Ethyl Cellulose

In order to compare the production rate of related materials according to the use of ethyl cellulose as a water-resistant coating material, the procedure of Example 2 was repeated except for not employing ethyl cellulose in the first coating layer to obtain the complex formulation of Comparative Example 7.

The oral complex formulations prepared in Comparative Example 7 and Example 2 were placed in sealed high-density polyethylene bottles and stored under accelerated storage condition (60° C.) for 1 week. Samples were taken, extracted with 80% acetonitrile (ACN), and then evaluated by HPLC analysis.

HPLC analysis was performed using a stainless steel column of approximately 250 mm length, packed with 5 μm C₁₈ or a similar column (Inertsil-ODS2, GL sciences) with a mobile phase of water:acetonitrile:1%(v/v) trifluoroacetic acid solution (62:37:1 (v/v)) at the flow rate of 0.75 mL/min. The peak of related material RRT 0.72 as compared to the major peak of rosuvastatin, was analyzed quantitatively. The results are shown in Table 8.

TABLE 8

| | Comp. Ex. 7 | Ex. 2 |
|---|---|---|
| Initial | 0.12 | 0.01 |
| After 1 week | 2.46 | 0.61 |

As shown in Table 8, Example 2 which used ethyl cellulose demonstrated an excellent inhibitory effect on the production of the related material as compared to Comparative Example 7 which did not use ethyl cellulose.

### Examples 9 and 10

### Preparation of Oral Complex Formulation

The procedure of Example 5 was repeated except for using magnesium hydroxide instead of sodium hydrogen carbonate in accordance with the ingredients described in Table 9, to obtain the oral complex formulations of Examples 9 and 10.

TABLE 9

| | mg/capsule | | |
|---|---|---|---|
| | Ingredient | Ex. 9 | Ex. 10 |
| Soft gelatin capsule | Omega-3 fatty acid ester | 1000.0 | 1000.0 |
| | Gelatin | 246.8 | 246.8 |
| | Sorbitol sorbitan soln. | 180.0 | 180.0 |
| | Glycine | 1.2 | 1.2 |
| The first coating layer | HPMC 2910 | 74.0 | 74.0 |
| | PEG 6000 | 10.0 | 10.0 |
| | Povidone | 10.0 | 10.0 |
| | Ethylcellulose | 28.0 | 28.0 |
| The second coating | Rosuvastatin calcium | 10.4 | 10.4 |
| layer | (rosuvastatin) | (10.0) | (10.0) |
| | NaHCO₃ | — | — |
| | CaCO₃ | — | — |
| | Mg(OH)₂ | 1.0 | 0.1 |
| | Kollicoat IR | 60.0 | 60.0 |
| | Povidone | 8.8 | 8.8 |

While the invention has been described with respect to the above specific embodiments, it should be recognized that various modifications and changes may be made to the invention by those skilled in the art which also fall within the scope of the invention as defined by the appended claims.

What is claimed is:

1. An oral complex formulation, which comprises:
(1) a capsule core comprising a hard or soft capsule containing sorbitol, sorbitan and, as a pharmaceutically effective component, an omega-3 fatty acid;
(2) a first coating layer comprising a water-resistant coating material, said coating layer being formed on the surface of the capsule core; and
(3) a second coating layer comprising an HMG-CoA reductase inhibitor and a basic stabilizer, said second coating layer being formed on the surface of the first coating layer.

2. The oral complex formulation of claim 1, wherein said sorbitol is in an amount ranging from 1 to 40% by weight, and said sorbitan is in an amount ranging from 1 to 45% by weight, based on the total weight of the hard or soft capsule.

3. The oral complex formulation of claim 1, wherein said hard or soft capsule comprises glycerol or its derivative in an amount of at most 20% by weight, based on the total weight of the capsule.

4. The oral complex formulation of claim 3, wherein said glycerol or its derivatives is propylene glycol, polyethylene glycol, medium-chain triglyceride oils, or a derivative thereof.

5. The oral complex formulation of claim 1, wherein said omega-3 fatty acid is selected from the group consisting of natural or synthesized omega-3 fatty acid; pharmaceutically acceptable esters, derivatives, precursors or salts thereof; and any mixture of the foregoing.

6. The oral complex formulation of claim 5, wherein said ester is selected from the group consisting of mono-, di- and triglycerides, and methyl and ethyl esters of omega-3 fatty acids; and the derivative is selected from the group consisting of polysaccharide derivatives and polyoxyethylene derivatives of omega-3 fatty acids.

7. The oral complex formulation of claim 5, wherein said omega-3 fatty acid is selected from the group consisting of eicosapenta-5,8,11,14,17-enoic acid (eicosapentaenoic acid, EPA), docosahexa-4,7,10,13,16,19-enoic acid (docosahexaenoic acid, DHA), and/or α-linolenic acid and precursors thereof.

8. The oral complex formulation of claim 1, wherein said omega-3 fatty acid is in an amount ranging from 70 to 95% by weight based on the total weight of the capsule core.

9. The oral complex formulation of claim 1, wherein said water-resistant coating material is selected from the group consisting of hydroxyethyl cellulose, hydroxypropyl cellulose, hydroxypropyl methylcellulose, polyvinylpyrrolidone, polyvinylpyrrolidone-vinyl acetate copolymer, ethyl cellulose and a mixture thereof.

10. The oral complex formulation of claim 1, wherein said water-resistant coating material comprises ethyl cellulose.

11. The oral complex formulation of claim 1, wherein said water-resistant coating material is in an amount ranging from 15 to 75% by weight based on the total weight of the first coating layer.

12. The oral complex formulation of claim 1, wherein said HMG-CoA reductase inhibitor is selected from the group consisting of simvastatin, pravastatin, fluvastatin, atorvastatin, cerivastatin, rosuvastatin, pitavastatin, and pharmaceutically acceptable salts thereof.

13. The oral complex formulation of claim 1, wherein said HMG-CoA reductase inhibitor is in an amount ranging from 0.05 to 20 parts by weight based on 100 parts by weight of the capsule core.

14. The oral complex formulation of claim 1, wherein said basic stabilizer is selected from the group consisting of magnesium carbonate (MgCO₃), sodium hydrogen carbonate (NaHCO₃), magnesium hydroxide (Mg(OH)₂), and a mixture thereof.

15. The oral complex formulation of claim 1, wherein said basic stabilizer is in an amount ranging from 0.01 to 40% by weight based on the total weight of the second coating layer.

16. The oral complex formulation of claim 15, wherein said basic stabilizer is magnesium carbonate, and the amount thereof is in the range of 5 to 40% by weight based on the total weight of the second coating layer.

17. The oral complex formulation of claim 15, wherein said basic stabilizer is sodium hydrogen carbonate, and the amount thereof is in the range of 0.01 to 2% by weight based on the total weight of the second coating layer.

18. The oral complex formulation of claim 15, wherein said basic stabilizer is magnesium hydroxide, and the amount thereof is in the range of 0.01 to 2% by weight based on the total weight of the second coating layer.

19. The oral complex formulation of claim 1, wherein said second coating layer comprises the HMG-CoA reductase inhibitor and the basic stabilizer in a weight ratio of 0.1:1 to 200:1.

20. The oral complex formulation of claim 19, wherein said basic stabilizer is magnesium carbonate, and the HMG-CoA reductase inhibitor and magnesium carbonate has a weight ratio of 0.1:1 to 3.0:1.

21. The oral complex formulation of claim 19, wherein said basic stabilizer is sodium hydrogen carbonate, and the HMG-CoA reductase inhibitor and sodium hydrogen carbonate has a weight ratio of 10:1 to 100:1.

22. The oral complex formulation of claim 19, wherein said basic stabilizer is magnesium hydroxide, and the HMG-CoA reductase inhibitor and magnesium hydroxide has a weight ratio of 10:1 to 100:1.

23. The oral complex formulation of claim 1, wherein said second coating layer comprises a coating material selected from the group consisting of hydroxypropylmethyl cellulose, polyvinylpyrrolidone, polyethylene glycol, polyvinyl alcohol-polyethylene glycol graft copolymer, and a mixture thereof.

24. The oral complex formulation of claim 1, wherein said oral complex formulation comprises the first coating layer in an amount ranging from 1 to 20 parts by weight, and the second coating layer in an amount ranging from 3 to 30 parts by weight, based on 100 parts by weight of the capsule core.

25. A method for preparing the oral complex formulation of claim 1, which comprises the steps of:
i) filling a hard or soft capsule comprising sorbitol and sorbitan with an omega-3 fatty acid to prepare a capsule core;
ii) forming a first coating layer comprising a water-resistant coating material on the surface of the core; and
iii) forming a second coating layer comprising an HMG-CoA reductase inhibitor and a basic stabilizer on the surface of the first coating layer.
